(19) United States
(12) Patent Application Publication
(10) Pub. No.: US 2013/0165419 A1
(43) Pub. Date: Jun. 27, 2013

(54) COMBINATION ANTI-INFLAMMATORY OPHTHALMIC COMPOSITIONS

(71) Applicants: Richard L. Lindstrom, Wayzata, MN (US); Kamran Hosseini, Hayward, CA (US); Lyle M. Bowman, Pleasanton, CA (US)

(72) Inventors: Richard L. Lindstrom, Wayzata, MN (US); Kamran Hosseini, Hayward, CA (US); Lyle M. Bowman, Pleasanton, CA (US)

(73) Assignee: INSITE VISION INCORPORATED

(21) Appl. No.: 13/333,534

(22) Filed: Dec. 21, 2011

(51) Int. Cl. A61K 31/573; A61P 27/02; A61P 31/12; A61P 31/04; A61P 27/12; A61P 27/06; A61P 35/00; A61P 33/02; A61P 31/10; A61P 29/00; A61P 27/14

(52) U.S. Cl. 514/171

(57) ABSTRACT

Compositions and systems for topical ophthalmic application, which include an aqueous mixture of steroidal and non-steroidal anti-inflammatory agents in a flowable mucoadhesive polymer, for treating inflammation and inflammatory conditions of the eye.

### FIELD OF THE INVENTION

The present invention relates to ophthalmic formulations; more particularly to ophthalmic formulations employing combined non-steroidal anti-inflammatory and steroidal anti-inflammatory agents.

### BACKGROUND OF THE INVENTION

A variety of diseases and disorders of the eye are associated with and/or indicated by inflammation of the eye including but not limited to, scleritis, episcleritis, dry eye, blepharitis, conjunctivitis, and uveitis, including iritis, cyclitis, retinitis, and choroiditis. Inflammation of the eye can also occur due to trauma to the eye or post-operatively, such as after cataract surgery or laser surgery, for example.

Treatment of inflammation of the eye can involve frequent dosing regimens which can erode patient compliance. From a delivery perspective, further challenges include formulating ophthalmic vehicles at viscosities low enough for reliable administration in drop form without impacting delivery efficiency and, at the same time, maintaining sufficient viscosity and mucoadhesion so that the delivered medicament remains in or on the ocular surface for a sufficient period of time to effectively treat the inflamed eye. Drug delivery to the ocular surface and mucosa faces the additional obstacle of various clearance mechanisms present in the eye.

### SUMMARY OF THE INVENTION

The present invention provides topical ophthalmic formulations containing a combination non-steroidal anti-inflammatory agent and steroidal anti-inflammatory agent in an ophthalmically acceptable vehicle comprising a flowable mucoadhesive polymer. It has been discovered that the compositions of the invention not only facilitate a slow release of the combined agents over a prolonged period of time, but also facilitates a high absorption and retention of the combination of agents by the aqueous humor of the eye. From a biological point of view, among others, the anti-inflammatory components of the present invention interact and interfere with a collection of different inflammatory mediators and pathways, providing a superior anti-inflammatory action.

In some aspects, embodiments disclosed herein relate to an ophthalmic composition comprising a therapeutically effective amount of bromfenac, a therapeutically effective amount of a steroidal anti-inflammatory, and an opthalmically acceptable vehicle comprising a flowable mucoadhesive polymer, wherein the composition has a viscosity formulated for administration to the eye of a mammal in drop form.

In some aspects, embodiments disclosed herein related to a method for therapeutic treatment of an inflammatory condition of the eye in a mammal comprising: (a) providing an ophthalmic composition comprising a therapeutically effective amount of bromfenac, a therapeutically effective amount of a steroidal anti-inflammatory, and an opthalmically acceptable vehicle comprising a flowable mucoadhesive polymer, wherein the composition has a viscosity formulated for administration to the eye of a mammal in drop form; and (b) administering the composition to the eye of a mammal to treat inflammation or inflammatory conditions of the eye.

It is to be understood that both the foregoing general description and the following detailed description are exemplary and explanatory only and are not restrictive of the invention, as claimed.

### DETAILED DESCRIPTION OF THE INVENTION

The present invention provides topical ophthalmic formulations containing a non-steroidal anti-inflammatory agent (NSAID) and a steroidal anti-inflammatory agent in an ophthalmically acceptable vehicle. The combination therapy can provide control of inflammation via reduced dosages of each of the individual components, compared to typical dosing of a single agent, thus reducing the side effects of any one agent. For example, typical dosing of ophthalmic steroidal agents alone can result in elevated intraocular pressure (IOP) and can slow healing of wounds in the eye. Typical dosing regimens of NSAIDs, on the other hand, can cause keratitis. In some patients, NSAID usage can result in epithelial breakdown, corneal thinning, erosion, ulceration, and/or perforation. The side effects due to either agent alone can reduce the period of time over which the drug can be administered, while inflammation, or the underlying cause of inflammation, may persist.

Non-steroidal anti-inflammatory agents are known to inhibit cyclooxygenases, enzymes associated with pain and inflammation in mammals. Cyclooxygenases are essential in the biosynthesis of prostaglandins, which have been shown in many animal models to be mediators of intraocular inflammation. Although steroidal compounds have been used to treat such inflammation, non-steroidal anti-inflammatory agents from the group of drugs known as cyclooxygenase inhibitors have been substituted for steroids because they have not shown the same propensity to produce side-effects in ocular tissues as compared to ophthalmic steroids. Non-steroidal agents are also widely prescribed to reduce pain and inflammation in a wide number of tissues. When used as topical agents in the eye, they suppress inflammatory responses and have been shown to prevent particular side-effects of surgical trauma (on the pupil preventing surgical meiosis), fluid accumulating in the back of the eye after cataract surgery (post-surgical macular edema) and the appearance of inflammatory cells and vessel leakage in the anterior chamber. Topical application of non-steroidal anti-inflammatory agents in the eye also appears to relieve some of the itching due to allergic conjunctivitis. Diclofenac sodium, suprofen, and flurbiprofin are non-steroidal anti-inflammatory agents that have been used for the treatment of postoperative inflammation in patients who have undergone cataract extraction.

In some embodiments, the present invention provides topical ophthalmic formulations containing the NSAID bromfenac in conjunction with a steroidal anti-inflammatory agent in an ophthalmically acceptable vehicle. In some such embodiments, the steroidal anti-inflammatory agent is a glucocorticoid. Bromfenac can be advantageously used in lieu of other NSAIDs in a reduced interval regimen, aiding in patient compliance. Bromfenac is commonly used to treat patients who have undergone cataract removal. The chemical structure of bromfenac is disclosed in U.S. Pat. No. 4,910,225, which is hereby incorporated in its entirety by reference. A sterile ophthalmic solution of bromfenac as sodium salt equivalent to 0.09% bromfenac free acid is currently marketed as Bromday® by ISTA/Senju Pharmaceuticals with a recommended dosing schedule of 1 drop per 24 hours. However, the above mentioned formulation has been shown not to provide a good control of prostaglandin-mediated inflammation when compared to ketorolac (another NSAID) partly due to its concentration drop in the eye after twelve hours, consistent with its on-label dosing schedule (Bucci et al., J Cataract Refract Surg. 34(9):1509-12 (2008)). The present invention employing a glucocorticoid in combination with bromfenac can address control of prostaglandin-mediated inflammation. Another potential benefit of the combination therapy of the invention is that the blockage of the cyclooxygenase pathway by a NSAID could amplify the generation of leukotrienes which causes an increase in chemotaxis of inflammatory cells resulting in enlarged cellular infiltration in ocular tissues unless this response is effectively suppressed by a steroid.

In some embodiments, the present invention provides topical ophthalmic formulations containing the NSAID bromfenac in conjunction with the glucocoritoid dexamethasone, in particular. In accordance with embodiments of the invention, this combination has been found to be particularly effective in the treatment of inflammation. In particular, dexamethasone can advantageously exhibit its anti-inflammatory effects at a relatively basic pH where bromfenac can also be beneficially administered. Furthermore, the present invention can provide synergistic effects for the (partial) relief of macular edema, which has been shown to be responsive for both corticosteroids and NSAIDs.

In some embodiments, the present invention further includes administration of the combination therapy in an ophthalmically acceptable vehicle comprising a flowable mucoadhesive polymer. It has been indicated that compositions of the invention incorporating such carrier vehicles exhibit a slow release of these agents over a prolonged period of time facilitating a higher absorption and retention of the agents by the aqueous humor of the eye. The increased absorption and retention of steroidal and non-steroidal agents in the eye, imparted by ophthalmic vehicles disclosed herein, allows subjects to apply fewer doses to treat the affected eye. Reduced dosing increases convenience of using the combination formulation, while a higher absorption and retention of the agents in the eye increases their efficacy in treating inflammation and inflammatory conditions of the eye. In general, it has been shown that control over dosing and concentration of NSAIDs delivered to the aqueous humor has been correlated to a higher efficacy in treating inflammation of the eye (Bucci et al., J Cataract Refract Surg. 34(9):1509-12 (2008)).

In some embodiments, the present invention provides topical ophthalmic formulations containing bromfenac and dexamethasone in a flowable mucoadhesive polymer that reduce the required dosing of these agents to, for example, once per day administration while facilitating the absorption and retention of a higher concentration of these agents in the the eye and/or its related or surrounding tissues such as, for example, retina to treat prostaglandin-mediated inflammation and/or other inflammatory conditions of the eye. Given the guidance provided herein other advantages will be apparent to the skilled artisan.

As used herein the term “ophthalmic composition” refers to a composition intended for application to the eye or its related or surrounding tissues such as, for example, eyelid. The term also includes compositions intended to therapeutically treat conditions of the eye itself or the tissues surrounding the eye and compositions administered via the ophthalmic route to treat therapeutically a local condition other than that involving the eye. The ophthalmic composition can be applied topically or by other techniques, known to persons skilled in the art, such as injection to the eye or its related tissues. Examples of suitable topical administration to the eye include administration in eye drops and by spray formulations. A further suitable topical administration route is by subconjunctival injection. The agents can also be provided to the eye periocularly or retro-orbitally. Although it is an advantage of the invention that intracameral administration is not required, this and other routes of administration are not outside the scope of the invention.

As used herein an “ophthalmically acceptable vehicle” is one which allows delivery of a medicament to the eye and/or eyelids, to treat an ocular disease or condition without deleterious effects on the eye. An ophthalmically acceptable vehicle is one that can maintain proper intraocular pressure and provide solutions of medicaments that are isotonic, mildly hypotonic, or mildly hypertonic. To maintain such conditions one can include various non-ionic osmolality-adjusting compounds such as polyhydric alcohols, including for example, glycerol, mannitol, sorbitol, or propylene glycol. Alternatively, osmolality adjusting compounds can include ionic salts such as sodium or potassium chloride. An ophthalmically acceptable vehicle can also include buffers to adjust the vehicle to an acceptable pH, which can range from about 3 to 6.5, and in some embodiments from about 4 to 8, including any pH in between. Compositions of the present invention including bromfenac can have a pH at the upper end of this scale as described herein. Such buffer systems include, but not limited to, acetate buffers, citrate buffers, phosphate buffers, borate buffers and mixtures thereof. Specific buffer components useful in the present invention include. but not limited to, citric acid/sodium citrate, boric acid, sodium borate, sodium phosphates, including mono, di- and tri-basic phosphates, such as sodium phosphate monobasic monohydrate and sodium phosphate dibasic heptahydrate, and mixtures thereof. It should be noted that any other suitable ophthalmically acceptable buffer components can be employed to maintain the pH of the ophthalmic formulation so that the ophthalmic formulation is provided with an acceptable pH, and the foregoing buffer components are merely exemplary examples of such buffer components.

As used herein “an ophthalmically acceptable salt” include those that exhibit no deleterious effects on the eye as well as being compatible with the active ingredient itself and the components of the ophthalmically acceptable vehicle. Salts or zwitterionic forms of a medicament can be water or oil-soluble or dispersible. The salts can be prepared during the final isolation and purification of the medicament or separately by adjusting the pH of the appropriate medicament formulation with a suitable acid or base. An ophthalmically acceptable salt can also include the aforementioned buffer systems.

As used herein, the term “carboxyl-containing polymer” refers to a polymer that contains the carboxylic acid functional group. This functional group can be substantially ionized, for example, and exist as a carboxylate anion (COO⁻), rendering the polymer negatively charged. In the context of an ophthalmically acceptable vehicle, the degree of ionization can depend on the pH, which is mediated by any buffer system, and the presence other components in the vehicle that contain Lewis basic atoms, such as an amine-functionalized polymer. A Lewis base is donor of a pair of electrons and as such, is capable of accepting hydrogen ion (H⁺) from a carboxyl group (COOH).

As used herein, the term “cationic polymer” refers to a positively-charged, amine-functionalized polymer. The polymer contains nitrogen atoms that are quaternized or capable of being quaternized upon adjustment to a sufficiently low pH and/or in the presence of a proton donor, such as the carboxyl containing polymer, or other Lewis acid (i.e. an electron pair acceptor). A quaternized nitrogen atom is a nitrogen atom engaged in bonding to four other atoms, thus causing nitrogen to have a net formal charge of plus one (+1). Examples of nitrogen atoms carrying positive charge include, but not limited to, NR₄⁺, NR₃H⁻, NR₂H⁺, NRH₂⁺, wherein R can represent any atom or group of atoms bonded to nitrogen.

As used herein “viscosity” refers to a fluid's resistance to flow. The unit of viscosity is dyne second per square centimeter [dyne·s/cm²], or poise [P]. This type of viscosity is also called dynamic viscosity, absolute viscosity, or simple viscosity. This is distinguished from kinematic viscosity which is the ratio of the viscosity of a fluid to its density.

As used herein “mucoadhesive” or “mucoadhesion” refers to the ability of the ophthalmically acceptable vehicle to adhere to the ocular mucosa. Mucoadhesive agents used in the invention include carboxy-containing polymers capable of forming hydrogen bonds. Mucoadhesion can depend on pH and the density of hydrogen bonding groups. In the vehicle of the present invention, the density of cross-linking in the carboxy-containing polymer can affect mucoadhesion. Thus, a lightly cross-linked polymer system has sufficient flexibility to form multiple hydrogen bonds, making it a good mucoadhesive agent. Another vehicle component that can affect mucoadhesion is the presence of a secondary polymer, which can interact with the carboxy-containing polymer, as explained further below.

As used herein the term “flowable mucoadhesive polymer” refers to a carboxy-containing polymer, e.g., lightly crosslinked polymers of acrylic acid or the like, having an optimal in vivo mucosal absorption rate, safety, degradability and flowability for an eye drop. The flowable mucoadhesive polymers used in the present invention are water insoluble, water-swellable, biodegradable polymer carriers including lightly crosslinked carboxy-containing polymers such as polycarbophil (Noveon® AA-1, Lubizol Corp., Wickliffe, Ohio) or other Carbopol® polymers (Lubizol Corp., Wickliffe, Ohio). Suitable carboxy-containing polymers for use in the present invention and methods for making them are described in U.S. Pat. Nos. 5,192,535 to Davis et al. which is hereby incorporated in its entirety by reference. A suitable carboxy-containing polymer system for use in the present invention is known by the tradename DuraSite® (InSite Vision Inc., Alameda, Calif.), containing polycarbophil, which is a sustained release topical ophthalmic delivery system that releases drug at a controlled rate. DuraSite® encompass lightly crosslinked polymers that are prepared by suspension or emulsion polymerizing at least about 90% by weight of a carboxyl-containing monoethylenically unsaturated monomer such as acrylic acid with from about 0.1% to about 5% by weight of a polyfunctional, or difunctional, crosslinking agent such as divinyl glycol(3,4-dihydroxy-1,5-hexadiene), having a particle size of not more than about 50 μm in equivalent spherical diameter.

As used herein the term “lightly crosslinked polymer” encompasses any polymer prepared by suspension or emulsion polymerization having a main polymer backbone comprising at least about 90% by weight of the polymer with a crosslinking agent present in a range from about 0.1% to about 5% by weight of the polymer, including about 0.1%, about 0.2%, about 0.3%, about 0.5%, about 0.6%, about 0.7%, about 0.8%, about 0.9%, about 1.0%, about 1.5%, about 2.0%, about 2.5%, about 3.0%, about 3.5%, about 4.0%, about 4.5%, and about 5.0%, including any fractional amount in between. In some embodiments, the main polymer backbone comprises from about 90% to about 99.9% by weight of the polymer. In some embodiments, the main polymer backbone comprises about 90%, about 91%, about 92%, about 93%, about 94%, about 95%, about 96%, about 97%, about 98%, about 99%, about 99.5%, or about 99.9% by weight of the polymer, including any fractional amount in between. The main polymer backbone can comprise a single monomer unit or can be a copolymer comprising two, three, or any number of monomer units. At least one monomer unit of a main polymer backbone has a functional moiety capable of supporting a charge, such as a carboxyl group, a sulfate group, a phosphate group, and the like. The crosslinking agent may be any difunctional or polyfunctional crosslinking agent.

When formulated with an ophthalmic medicament, e.g., bromfenac, and bromfenac in combination with a glucocorticoid, such as dexamethasone, into solutions or suspensions in aqueous medium, the amount of lightly crosslinked polymer ranges from about 0.5% to about 1.5% by weight, based on the total weight of the aqueous suspension, the pH is from about 7.4 to about 8.5, and the osmotic pressure (osmolality or tonicity) is from about 10 mOsM to about 400 mOsM, provide new topical ophthalmic medicament delivery systems having suitably low viscosities which permit them to be easily administered to the eye in drop form, and hence be comfortably administrable in consistent, accurate dosages. The compositions of the invention containing DuraSite® will rapidly gel in the eye after coming into contact with the eye's tear fluid to a substantially greater viscosity than that of the originally-introduced suspension or solution and thus remain in place for prolonged periods of time to provide sustained release of the ophthalmic medicament.

As used herein, “administered to the eye” means that an ophthalmically acceptable vehicle, along with a medicament, is in the form of an eye drop that can be applied directly to the surface of the eye and/or in the eyelid margins, such administration techniques being familiar to persons skilled in the art.

As used herein the term “retained in or carried with” or “retaining or carrying” embraces generally all ways that the steroidal and nonsteroidal agents can be associated with the flowable mucoadhesive polymer. For example, they can be in aqueous solution dispersed throughout the polymer. A bromfenac concentration of up to about 0.25% can be in solution mixed with or dispersed throughout the flowable mucoadhesive polymer carrier. Bromfenac can also be in suspension with the polymer depending on its concentration. For example, when bromfenac is used in an amount more than about 0.36% by weight of the composition, some of the bromfenac can be in suspension with the polymer carrier while an amount of up to about 0.25% of bromfenac will still be in solution and mixed with the polymer carrier.

As used herein the term “inflammation or inflammatory conditions of the eye” refers to an ocular disease or any inflammatory condition of the eye and external tissues surrounding eye, e.g., eyelid, influenced by various exogenous or endogenous agents or events. Endogenous factors include, but are not limited to, inflammatory chemokines, cytokines, mediators, nuclear transcription factors, antigens, autogens or hormones that can cause acute or chronic inflammation, pain, redness, swelling, wateriness and itchiness of the eye or its surrounding tissues. Exogenous agents or events include, but are not limited to, infection, injury, radiation, surgery or damage to the eye or its surrounding tissues, which initiate biochemical reactions leading to an inflammation. An ocular disease is one caused by vascular leakage in the eye or by inflammation in the eye. Examples of conditions related to inflammation in the eye include, but are not limited to the following: surgical trauma; dry eye; allergic conjunctivitis; viral conjunctivitis; bacterial conjunctivitis; blepharitis; anterior uveitis; injury from a chemical; radiation or thermal burn; or penetration of a foreign body, signs and symptoms of eye problems (e.g., pain in or around the eye, redness especially accompanied by pain in the eye (with or without movement), extreme light sensitivity, halos (colored circles or halos around lights), bulging (protrusion) of the eye or swelling of eye tissues, discharge, crusting or excessive tearing; eyelids stuck together, especially upon awakening, blood inside the front of the eye (on the colored part) or white of the eye); cataracts; pain and inflammation associated with wearing contact lenses; corneal conditions (e.g., conjunctival tumor excision, conjunctivitis (“Pink Eye”), cornea edema after cataract surgery, corneal clouding, corneal transplantation, corneal ulcer, dry eye syndrome, dystrophies, conditions associated with excimer laser phototherapeutic keratectomy, herpes simplex keratitis, keratoconus, pterygium, recurrent erosion syndrome); eye movement disorders; glaucoma; ocular oncology, oculoplastics (e.g., cosmetic surgery, enucleation, eyelid and orbit injuries, ectropion, entropion, graves' disease, involuntary eyelid blinking); conditions associated with refractive surgery; and retinal conditions.

As used herein the term “sustained release delivery system” or “sustained release composition” refers to a composition comprising a flowable mucoadhesive polymer—which is a carboxy-containing polymer such as polycarbophil and DuraSite®, as described in U.S. Pat. No. 5,192,535—which facilitates a sustained release of the combination steroidal and NSAID agents of the invention. Such compositions may include other biologically active agents besides this combination. Typically, the sustained release compositions of the invention can contain from about 0.005% (w/w) to about 0.5% of NSAID. In an exemplary embodiment, the range of bromfenac loading can be in a range from about 0.01% (w/w) to about 0.2%. In another exemplary embodiment, the range of bromfenac loading can be in a range from about 0.045% (w/w) to about 0.09%. In still a further exemplary embodiment, bromfenac loading can be in a range from about 0.01% to about 0.25%. The sustained release delivery systems or compositions of this invention can be formed into many shapes such as a solution, a gel, a film, a pellet, a rod, a filament, a cylinder, a disc, a wafer, nanoparticles or a microparticle. A “microparticle” as defined herein, comprises a blend polymer component having a diameter of less than about one millimeter and having bromfenac dispersed therein. A microparticle can have a spherical, non-spherical or irregular shape. Typically, the microparticle will be of a size suitable for injection. In one embodiment, the size range for microparticles is from about one to about 50 microns in diameter.

As defined herein, a sustained release of a biologically active agent is a release of the biologically active agent, such as a combination of bromfenac and dexamethasone, from a sustained release delivery system or composition. The release occurs over a period which is longer than that period during which a therapeutically significant amount of the biologically active agent would be available following direct administration of a solution of the biologically active agent. In one embodiment, a sustained release occurs over a period of greater than six to twelve hours such as about twenty-four hours or longer. A sustained release of biologically active agent can be a continuous or a discontinuous release, with relatively constant or varying rates of release. The continuity of release and level of release can be affected by the type of polymer composition used (e.g., monomer ratios, molecular weight, and varying combinations of polymers), agent loading, and/or selection of excipients to produce the desired effect.

As used herein the term “treating” or “treatment” refers to reducing, ameliorating reversing, alleviating, inhibiting the progress of, or preventing a disease or a medical condition of the eye itself or the tissue surrounding the eye or the symptoms associated therewith. The term also encompasses prophylaxis, therapy and cure. The subject receiving “treatment,” or whom undergoes “treating” is any mammal in need of such treatment for (eye-related inflammation or inflammatory conditions), including primates, in such as humans, and other mammals such as equines, cattle, swine and sheep; and poultry and domesticated mammals and pets in general.

The term “therapeutically effective amount” as used herein means that the amount of a composition elicits a beneficial biological or medicinal response in a tissue, system, animal or human. For example, a therapeutically effective amount of a composition of the invention is a dose which leads to a clinically detectable improvement or treatment (as defined above) of the eye of a subject suffering from an inflammatory eye condition or disease. An “effective amount” when used in connection with treating an ocular disease or condition is intended to qualify the amount of a medicament used in the treatment of a particular ocular disease or condition. This amount will achieve the goal of preventing, reducing, or eliminating the ocular disease or condition. An effective amount depends on the particular active ingredient to be administered, although ophthalmic formulations can include, for example, from about 0.05% to about 5.0% by weight, while in other embodiments the active ingredient is present in a range from about 0.08% to about 0.12% by weight. 0.01 mg/ml to 100 mg/ml per dose in one embodiment and from about 1 to 50 mg/ml dose in another embodiment. An “effective amount” can include a dose regimen once per day, twice per day, thrice per day, and so on.

As used herein, the term “about” refers to an approximation of a stated value within an acceptable range, such as plus or minus about 5% of the stated value.

In some embodiments, the present invention provides ophthalmic compositions for treating inflammation and inflammatory conditions of the eye; such compositions include a non-steroidal anti-inflammatory agent, exemplified by bromfenac, and a steroidal anti-inflammatory agent, exemplified by dexamethasone, in an ophthalmically acceptable vehicle comprising a flowable mucoadhesive polymer to increase the retention of these anti-inflammatory agents in the eye for a longer period of time.

In some embodiments, the invention provides a sustained release delivery system for a non-steroidal anti-inflammatory agent and a steroidal anti-inflammatory agent in an ophthalmically acceptable vehicle comprising a flowable mucoadhesive polymer. In some such embodiments, the present invention provides a sustained release delivery system for topical ophthalmic delivery of bromfenac and dexamethasone.

In some embodiments, the present invention provides a bromfenac and dexamethsone composition in sustained release ophthalmic delivery systems suitable for administration at intervals of once daily or less often, such as, once per every two to three days.

In still other embodiments, the present invention provides a method for convenient therapeutic treatment using a composition or a delivery system including bromfenac and dexamethasone in an ophthalmically acceptable vehicle comprising a flowable mucoadhesive polymer, which has a prolonged release time for bromfenac and dexamethasone while facilitating a high absorption and retention of bromfenac and dexamethasone in the eye over the release-time period.

In still further embodiments, the present invention provides methods for treating, ameliorating or reducing inflammation or inflammatory conditions of the eye by providing a composition or a delivery system containing bromfenac and dexamethasone in an ophthalmic vehicle comprising a flowable mucoadhesive polymer, which has a prolonged release time for bromfenac and dexamethasone, while facilitating a high absorption and retention of bromfenac and dexamethasone by the eye over the release time period.

In some embodiments, the present invention provides a method of treating inflammation or inflammatory conditions of the eye or an ocular disease, injury or disorder in a patient by administering a topical ophthalmic formulation described herein. Furthermore the method may include a dosing regime of once, to two times daily administration into the eye to treat the pain and/or inflammation associated with the ocular disease, injury or disorder. In one embodiment, the dosing is a once a day bromfenac-dexamethasone formulation.

In some embodiments, methods are provided to treat an ocular disease, injury or disorder caused by surgery, physical damage to the eye, glaucoma, macular degeneration, or diabetic retinopathy. The formulation of bromfenac and dexamethasone, in accordance with the compositions and methods of the invention, can be used to treat retina conditions (e.g., macular edema, macular degeneration, etc.) since topical application of the inventive compositions results in high concentrations of the drugs bromfenac and dexamethasone in the retina. A still further aspect of the present invention is to use the bromfenac-dexamethasone formulations of the invention to treat ocular disease, injury or disorder wherein the ocular disease, injury or disorder is one caused by vascular leakage in the eye or by inflammation in the eye. Examples of conditions related to inflammation in the eye include, but are not limited to the following: surgical trauma; dry eye; allergic conjunctivitis; viral conjunctivitis; bacterial conjunctivitis; blepharitis; anterior uveitis; injury from a chemical; radiation or thermal burn; injury from penetration of a foreign body, pain in or around the eye, redness especially accompanied by pain in the eye; light sensitivity; seeing halos (colored circles or halos around lights); bulging (protrusion) of the eye; swelling of eye tissues; discharge, crusting or excessive tearing; eyelids stuck together, blood inside the front of the eye (on the colored part) or white of the eye); cataracts; pain and inflammation associated with wearing contact lenses; corneal-associated condition; conjunctival tumor excision; conjunctivitis known as Pink Eye; cornea edema after cataract surgery; corneal clouding; corneal transplantation; corneal ulcer; dry eye syndrome; dystrophies; condition associated with excimer laser phototherapeutic keratectomy; herpes simplex keratitis; keratoconus; pterygium; recurrent erosion syndrome; eye movement disorder; glaucoma; ocular oncology; oculoplastic condition resulted from cosmetic surgery, enucleation, eyelid and orbit injuries, ectropion, entropion, graves' disease, involuntary eyelid blinking; condition associated with refractive surgery; and retinal condition.

Retinal conditions include, but are not limited to, age related macular degeneration, AIDS-related ocular disease (e.g., CMV retinitis), birdshot retinochoroidopathy (BR), choroidal melanoma, coats disease, cotton wool spots, diabetic retinopathy, diabetic macular edema, cystoid macular edema, lattice degeneration, macular disease (e.g., macular degeneration, hereditary macular dystrophy, macular edema, macular hole, macular pucker, central serous chorioretinopathy), ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis (affecting retina) and uveitis.

Bromfenac is a non-steroidal anti-inflammatory agent which has the structural formula of

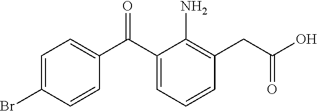 [SMILES: Nc1c(CC(=O)O)cccc1C(=O)c1ccc(Br)cc1]

The above compound to be used in accordance with the invention may be in a salt form or a hydrated form or both. The salt forms include alkali metal salts such as sodium salt and potassium salt, alkaline earth metal salts such as calcium salt and magnesium salt, among others, and any salt may suitably be used, provided that it can attain the object of the invention. The hydrated forms include monohydrate, sessquihydrate (1.5 H₂O), dihydrate, pentahydrate and any other hydrate forms may suitably be used, provided that it can attain the object of the invention.

In some embodiments, a steroidal anti-inflammatory agent consists essentially of a glucocorticoid. Glucocorticoids are potent anti-inflammatory agents and can often be successfully administered independent of the underlying cause of inflammation. Without being bound by theory, glucocorticoids' primary anti-inflammatory mechanism are reported to be related to lipocortin-1 (annexin-1) synthesis. Lipocortin-1 suppresses phospholipase A2, thereby blocking eicosanoid production, and inhibits various leukocyte inflammatory events. In addition, glucocorticoids have been shown to suppress cyclooxygenases, including COX-1 and COX-2. SEGRA's, or Selective Glucocorticoid Receptor Agonists may alos be used here as potent anti-inflammatory agents.

Glucocorticoids can initiate an anti-inflammatory effect by binding to the cytosolic glucocorticoid receptor (GR). After binding GR, the receptor-ligand complex translocates to the cell nucleus, where it can bind to glucocorticoid response elements (GRE) in the promoter region of target genes. The proteins encoded by these upregulated genes have a wide range of effects including anti-inflammatory effects mediated, for example, by lipocortin I as described above. Glucocorticoids can also reduce the transcription of pro-inflammatory genes by a mechanism of transrepression. Thus, inflammation associated with blepharitis can be ameliorated by glucocorticoid treatment.

Accordingly, in some embodiments, the steroidal anti-inflammatory present in compositions and methods of the invention includes a glucocorticoid including, for example, hydrocortisone, cortisone acetate, prednisone, prednisolone, methylprednisolone, dexamethasone, betamethasone, triamcinolone, beclomethasone, and fluorometholone. In some embodiments, the glucocorticoids include, for example, 21-acetoxypregnenolone, alclometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinolone hexacetonide, difluprednate their opthalmically acceptable salts, combinations thereof, and mixtures thereof. In one embodiment, the glucocorticoid includes dexamethasone, prednisone, prednisolone, methylprednisolone, medrysone, triamcinolone, loteprednol etabonate, opthalmically acceptable salts thereof, combinations thereof, and mixtures thereof.

In accordance with various embodiments of the invention, dexamethasone includes, for example, dexamethasone sodium phosphate, dexamethasone (alcohol), dexamethasone acetate, dexamethasone dimethylbutyrate, dexamethasone trimethylacetate, dexamethasone dipropionate, dexamethasone acefurate, and mixtures thereof.

In some embodiments, the steroidal anti-inflammtory, exemplified by dexamethasone, is present in a range from about 0.025% to about 0.25% by weight of the compostion. In some embodiments, the steroidal anti-inflammtory agent is present in a range from about 0.050% to about 0.1%. In some embodiments, the steroidal anti-inflammtory agent is present at about 0.025%, about 0.050%, about 0.075%, about 0.100%, about 0.125%, about 0.150%, about 0.175%, about 0.200%, about 0.225%, about 0.250%, including any value in between.

In some embodiments, according to any of the above aspects of the invention, the bromfenac content of the compositions of the invention is about 0.005% to about 0.5% by weight of the composition. In another embodiment, the bromfenac content of the compositions is about 0.01% to about 0.2% by weight of the composition. In another embodiment, the bromfenac content of the compositions is about 0.01% to about 0.09% by weight of the composition. In another embodiment, the bromfenac content of the compositions is about 0.045% to about 0.09% by weight of the composition

In some embodiments, according to any of the above aspects of the invention, the compositions of the invention have a pH in a range from about 7.4 to about 8.5; in other embodiments the pH is about 8.3. In some embodiments, the pH is about 8.0, about 8.1, about 8.2, about 8.3, about 8.4, or about 8.5.

The present invention also provides kits including a composition having bromfenac and dexamethasone in an ophthalmic carrier comprising a flowable mucoadhesive polymer for application to the eye of a mammal. The kit further includes instructions for how use the composition, eye dropper and other useful paraphanalia for topical delivery to the eye. The kit can provide the active anti-inflammatory agents as solids with a sterile aqueous solution to mix real time, or can provide the agents pre-mixed in the carrier vehicle.

In one embodiment, according to any of the above aspects of the invention, the viscosity of the compositions of the invention is in the range of about 1,000 to about 2,000 cps. In another embodiment, the viscosity of the composition of the invention is about 1,500 cps. When formulated as a topical ophthalmic delivery system, the viscosity of the composition of the invention is desirably in a range suitable for administration to the eye in drop form, such as a viscosity from about 1,000 to about 2,000 cps.

In one embodiment, according to any of the above aspects of the invention, the bromfenac is retained in or carried with the flowable mucoadhesive polymer. The flowable mucoadhesive polymer increases the retention of bromfenac in the eye for a longer period of time. In another embodiment, the entire bromfenac content of the composition of the invention is in aqueous solution.

In one embodiment, according to any of the above aspects of the invention, a percentage of bromfenac content of the compositions of the invention is in aqueous solution with the polymer while the remaining bromfenac remains in suspension with the polymer. In another embodiment, the bromfenac mixed with the polymer carrier can be in suspension to act as a reservoir established in suspension at the pH of the formulation. The amount established in suspension may vary depending on therapeutic needs, but it will be at least an amount sufficient to have a therapeutic effect on the eye upon delayed release from the suspension over a period of time. A sufficient amount of agent will also be present in solution to have an immediate therapeutic effect upon topical ophthalmic application. For example, about 80% to about 90% of the total bromfenac contained in the mixture will be in suspension, but this may vary depending on how much of the agent is desired for sustained delivery and the duration of delivery desired. The amount of bromfenac in suspension may, for instance, range from about 70% to about 99% or about 10% to about 99% by weight of the total amount of bromfenac contained in the mixture. The compositions will not, however, have 100% of bromfenac in suspension. Some amount will be in solution to provide the immediate therapeutic effect. In certain embodiments, the concentration of bromfenac and the pH of the composition is selected to ensure that a sufficient amount of bromfenac is in suspension to provide a therapeutic effect upon delayed deliver. In this way, the portion of the agent in solution is immediately available for therapeutic effect, while the portion in suspension serves as a reservoir and is released slowly over time.

In some embodiments, according to any of the above aspects of the invention, the flowable mucoadhesive polymer content of the composition of the invention is about 0.5% to about 1.5% by weight of the composition. In other embodiments, the flowable mucoadhesive polymer content of the composition of the invention is about 0.8% to about 1.0% by weight of the composition. In another embodiment, the flowable mucoadhesive polymers of the invention are crosslinked carboxy-vinyl polymers as carboxy-containing polymers. Suitable carboxy-containing polymers for use in the present invention and method for making them are described in U.S. Pat. No. 5,192,535 to Davis et al. which is hereby incorporated, in its entirety, by reference and relied upon. These polymer carriers include lightly crosslinked carboxy-containing polymers such as polycarbophil, or Carbopols®, dextran, cellulose derivatives, polyethylene glycol 400 and other polymeric demulcents such as polyvinylpyrolidone, polysaccaride gels and Gelrite®. In another embodiment, a carboxy-containing polymer system known by the tradename DuraSite® is used. DuraSite® is a lightly crosslinked polymer containing polycarbophil which is a sustained release topical ophthalmic delivery system that releases the drug at a controlled rate.

The lightly crosslinked polymers of acrylic acid or the like used in practicing this invention are, in general, well known in the art. In one embodiment such polymers are ones prepared from at least about 90% or from about 95% to about 99.9% by weight, based on the total weight of monomers present, of one or more carboxyl-containing monoethylenically unsaturated monomers. Acrylic acid is a carboxyl-containing monoethylenically unsaturated monomer, but other unsaturated, polymerizable carboxyl-containing monomers, such as methacrylic acid, ethacrylic acid, .beta.-methylacrylic acid (crotonic acid), cis-.alpha.-methylcrotonic acid (angelic acid), trans-.alpha.-methylcrotonic acid (tiglic acid), .alpha.-butylcrotonic acid, .alpha.-phenylacrylic acid, .alpha.-benzylacrylic acid, .alpha.-cyclohexylacrylic acid, .beta.-phenylacrylic acid (cinnamic acid), coumaric acid (o-hydroxycinnamic acid), umbellic acid (p-hydroxycoumaric acid), and the like can be used in addition to or instead of acrylic acid.

Such polymers are crosslinked by using a small percentage, i.e., less than about 5%, such as from about 0.5% or from about 0.1% to about 5%, or from about 0.2% to about 1%, based on the total weight of monomers present, of a polyfunctional crosslinking agent. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallylmethacrylamide and the like. Also included are polyalkenyl polyether crosslinking agents containing two or more alkenyl ether groupings per molecule, or alkenyl ether groupings containing terminal H₂C═C< groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053, which incorporated herein by reference in its entirety. Diolefinic non-hydrophilic macromeric crosslinking agents having molecular weights of from about 400 to about 8,000, such as insoluble di- and polyacrylates and methacrylates of diols and polyols, diisocyanate-hydroxyalxyl acrylate or methacrylate reaction products, and reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. Nos. 4,192,827 and 4,136,250, which incorporated herein by reference in its entirety.

The lightly crosslinked polymers can of course be made from a carboxyl-containing monomer or monomers as the sole monoethylenically unsaturated monomer present, together with a crosslinking agent or agents. They can also be polymers in which up to about 40%, or from about 0% to about 20% by weight, of the carboxyl-containing monoethylenically unsaturated monomer or monomers has been replaced by one or more non-carboxyl-containing monoethylenically unsaturated monomers containing only physiologically and ophthalmologically innocuous sub stituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat. No. 4,548,990, which incorporated herein by reference in its entirety, for a more extensive listing of such additional monoethylenically unsaturated monomers. In one embodiment, polymers are lightly crosslinked acrylic acid polymers wherein the crosslinking monomer is 2,3-dihydroxyhexa-1,5-diene or 2,3-dimethylhexa-1,5-diene.

The lightly crosslinked polymers used in practicing this invention are prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, or from about 3 to about 20 μm, in equivalent spherical diameter. In general, such polymers will range in molecular weight estimated to be about 250,000,000 to about 4,000,000,000 or about 500,000 to about 2,000,000,000.

According to any of the above aspects of the invention, the composition of the invention is an aqueous mixture that can also contain amounts of suspended lightly crosslinked polymer particles ranging from about 0.5% to about 1.5% by weight, or from about 0.8% to about 1.0% by weight, based on the total weight of the aqueous mixture. The aqueous mixture can be an aqueous solution of bromfenac and a flowable mucoadhesive polymer or an aqueous suspension of bromfenac and a flowable mucoadhesive polymer. In certain embodiments, the composition of the invention is prepared using pure, sterile water, such as deionized or distilled, having no physiologically or ophthalmologically harmful constituents, and is adjusted to a pH of from about 7.4 to about 8.5, in some embodiments from about 8.2 to about 8.4, and in other embodiments to a pH of about 8.3 using any physiologically and ophthalmologically acceptable pH adjusting acid, base or buffer, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylamino-methane), or the like and salts and buffers such as citrate/dextrose, sodium bicarbonate, ammonium chloride and mixtures of the aforementioned acids and bases. For example, bromfenac or its salt at may be dissolved and added by sterile filtration to a preparation containing sodium chloride, DuraSite® and surfactant. This mixture may then be adjusted to the appropriate pH by known techniques, for example by the addition of sodium hydroxide. Other methods will be apparent to one skilled in the art.

When formulating the composition of the invention as either an aqueous solution or an aqueous suspension, the osmolality can be adjusted to from about 10 mOsm/kg to about 400 mOsm/kg, using appropriate amounts of physiologically and ophthalmologically acceptable salts. Sodium chloride approximates physiologic fluid, and amounts of sodium chloride ranging from about 0.01% to about 1% by weight, or from about 0.05% to about 0.45% by weight, based on the total weight of the aqueous suspension, provide osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can also be used in addition to or instead of sodium chloride to achieve osmolalities within the above-stated ranges. Sugars like mannitol, dextrose, glucose or other polyols may be added to adjust the osmolality.

The amounts of flowable mucoadhesive polymer, the pH, and the osmotic pressure chosen from within the above-stated ranges are correlated with one another and with the degree of crosslinking of the polymer to give aqueous solutions or suspensions having viscosities ranging from about 1,000 to about 2,000 or 5,000 to about 20,000 cps respectively, as measured at room temperature (about 25° C.) using a Brookfield Digital LVT Viscometer equipped with a number 25 spindle and a 13R small sample adapter at 12 rpm. The compositions of the present invention have a viscosity that is suited for the selected route of administration. Alternatively, the visocisty can be 1000 to 3400 cps as measured with a Brookfield cone and plate viscosity DV-II+ with the spindle No. CP-52 at 6 rpm.

In one embodiment, according to any of the above aspects of the invention, the compositions of the present invention ordinarily contain one or more surfactants and, if desired, one or more adjuvants, including additional medicaments, buffers, antioxidants, tonicity adjusters, preservatives, thickeners or viscosity modifiers, and the like. Additives in the formulation may desirably include sodium chloride, EDTA (disodium edetate), and BAC (benzalkonium chloride) or sorbic acid, or both.

Compositions delivered by means of the sustained release medicament delivery system of this invention typically have residence times in the eye ranging from about 4 to about 8 hours. The bromfenac contained in these compositions is released from the composition at rates that depend on such factors as bromfenac itself and its physical form, the extent of drug loading and the pH of the system, as well as on any drug delivery adjuvants, such as ion exchange resins compatible with the ocular surface, which may also be present in the composition. In one embodiment, according to any of the aspects of the present invention, the composition of the invention provides a sustained concentration of bromfenac of between 10⁻⁸ and 10⁻⁴ M, in another embodiment between 10⁻⁷ and 10⁻⁵ M, in the aqueous or treated tissue of the eye for at least two hours, and in certain embodiments, at least three hours. In another embodiment, the composition of the invention provides sustained concentration of bromfenac of between 10⁻⁸ and 10⁻⁴ M, or between 10⁻⁷ and 10⁻⁵ M, in the aqueous or treated tissue of the eye for at least two hours, or at least three hours.

Ophthalmic compositions of the present invention may be formulated so that they retain the same or substantially the same viscosity in the eye that they had prior to administration to the eye. Alternatively, ophthalmic suspensions of the present invention may be formulated so that there is increased gelation upon contact with tear fluid. For instance, when a formulation containing DuraSite® is administered to the eye, the DuraSite® system swells upon contact with tears. This gelation or increase in gelation leads to a slower release rate of bromfenac, thereby extending the residence time of the composition in the eye. These events eventually leads to increased patient comfort, increase in the time bromfenac is in contact with the eye tissues, thereby increasing the extent of drug absorption and duration of action of the formulation in the eye.

The present invention further provides an ophthalmic vehicle with desirable rheological properties that are conducive to medicament delivery into the eye and provide corneal retention and in some such embodiments, comprise a modified Durasite® system. In some such embodiments, the vehicle uses a combination of an anionic carboxy-containing polymer in conjunction with a substantially smaller amount of a second polymer, for example, a cationic polymer. The second polymer is included at a sufficiently low concentration such that the particles of the carboxy-containing polymer remain suspended, yet when combined with the second polymer, the resulting vehicle has higher viscosity than the vehicle with the carboxy-containing polymer alone. The vehicle disclosed herein has the property that, when combined with tear fluid, its viscosity increases due to the higher pH of tear fluid. The resultant viscosity provides a means by which to increase the efficiency of medicament delivery and corneal retention.

The ophthalmically acceptable vehicle disclosed herein also has suitable mucoadhesive properties that can facilitate the absorption of poorly absorbed drugs by increasing the contact time of the drug with the ocular mucosa. Interactions between the vehicle and the ocular mucosa can include Van der Waals attractive forces, hydrogen bonding, and electrostatic interactions between the mucins of the ocular mucosa and the carboxy-containing polymer and the second polymer. Together, these forces can increase the residence time of a medicament in the eye. An additional benefit of the ophthalmically acceptable vehicle disclosed herein, is the ability to provide the medicament in a sustained release manner.

In one embodiment, the invention provides an ophthalmically acceptable vehicle that includes an aqueous suspension containing from about 0.1% to about 6.5% by weight, based on the total weight of the suspension, of a carboxyl-containing polymer prepared by polymerizing one or more carboxyl-containing monoethylenically unsaturated monomers and less than about 5% by weight of a crosslinking agent. The weight percentages of monomers are based on the total weight of monomers polymerized. The carboxyl-containing polymer has an average particle size of not more than about 25 μm in equivalent spherical diameter and is lightly cross-linked.

The vehicle further includes a second polymer, such as a cationic polymer, added in sufficient amount to increase the vehicle viscosity without the loss of polymer particle suspension, while still allowing the vehicle to be administered to the eye in drop form. Upon contact of the lower pH vehicle with higher pH tear fluid, the vehicle rapidly gels to a greater viscosity and therefore can remain in the eye for sustained release of a medicament contained within the vehicle.

In some embodiments, the ophthalmically acceptable vehicle uses carboxy-containing polymers in conjunction with a cationic polymer added in sufficient amount to increase the vehicle viscosity, while still allowing the carboxy-containing polymer particles to remain suspended. The vehicle can be in the form of a gel or liquid drops which release a medicament over time when administered to the eye. The carboxy-containing polymer is about 0.1 to about 6.5% in some embodiments, and, in other embodiments about 1.0 to about 1.3% by weight based on the total weight of the suspension of a cross-linked carboxy-containing polymer. Suitable carboxy-containing polymers are described, for example, in U.S. Pat. No. 5,192,535 to Davis et al. which is hereby incorporated in its entirety by reference. These polymer carriers include lightly crosslinked carboxy-containing polymers (such as polycarbophil, or CARBOPOLS®), dextran, cellulose derivatives, polyethylene glycol 400 and other polymeric demulcents such as polyvinylpyrolidone, polysaccaride gels and GELRITE®. A carboxy-containing polymer system known by the tradename DURASITE®, is a polycarbophil-based sustained release topical ophthalmic delivery system that can also be modified with such polymers disclosed herein.

In accordance with certain embodiments, an ophthalmically acceptable carrier capable of sustained release includes an aqueous suspension at a pH of from about 3 to about 8 and an osmolality of from about 10 to about 400 mOsm/kg containing from about 0.1% to about 6.5% by weight, based on the total weight of the suspension, of a carboxyl-containing polymer prepared by polymerizing one or more carboxyl-containing monoethylenically unsaturated monomers and less than about 5% by weight of a cross-linking agent, such weight percentages of monomers being based on the total weight of monomers polymerized. The suspension can have an initial viscosity of from about 1,000 to about 100,000 centipoises (cps). For example, the viscosity can be in a range from about 1,000 to about 5,000 cps, and in other embodiments from about 5,000 to about 10,000 cps, and in still other embodiments from about 10,000 to about 15,000 cps, and in still further embodiments from about 15,000 to about 20,000 cps, and in yet still further embodiments from about 50,000 to about 100,000 cps, including any values in between these recited values. The carboxy-containing polymer has average particle size of not more than about 50 μm, and in some embodiments, not more than about 30 μm, in equivalent spherical diameter. The polymer is lightly cross-linked to a degree such that although the suspension is administrable in drop form, upon contact of the lower pH suspension with the higher pH tear fluid of the eye, the suspension is gellable to a substantially greater viscosity than the viscosity of the suspension as originally administered in drop form. Accordingly, the resulting more viscous gel can remain in the eye for a prolonged period of time so as to release a medicament contained therein in sustained fashion. These properties remain upon addition of the second polymer to the carboxy-containing aqueous suspension. Without being bound by the theory, the cationic polymer increases the viscosity of the base carboxy-containing aqueous suspension, providing beneficial rheological and mucoadhesive properties.

The carboxy-containing polymer is, in one embodiment, prepared from at least about 50% by weight, and in other embodiments from at least about 90% by weight, of one or more carboxyl-containing monoethylenically unsaturated monomers. The carboxy-containing polymer can be prepared by suspension or emulsion polymerizing acrylic acid and a non-polyalkenyl polyether difunctional cross-linking agent to a particle size of not more than about 25 μm, in equivalent spherical diameter, in other embodiments. In one embodiment, the cross-linking agent is divinyl glycol. In other embodiments, up to about 40% by weight of the carboxyl-containing monoethylenically unsaturated monomers can be replaced by one or more non-carboxyl-containing monoethylenically unsaturated monomers containing only physiologically and ophthalmologically innocuous substituents.

The osmolality, in some embodiments, achieved by using a physiologically and ophthalmologically acceptable salt in an amount of from about 0.01% to about 1% by weight, based on the total weight of the suspensions. Exemplary salts include potassium and sodium chlorides and others as defined above.

In some embodiments, in a method of preparing sustained release topical ophthalmically acceptable vehicles, the foregoing suspensions modified with the cationic polymer, are prepared and packaged at the desired viscosity of from 1,000 to about 30,000 cps for administration to the eye in drop form. In one exemplary delivery method, the foregoing suspensions, containing the medicament, are administered to the eye at the initial viscosity in drop form to cause the administered suspension, upon contact with the higher pH tear fluid of the eye, to rapidly gel in situ to a significantly greater viscosity. The more viscous gel remains in the eye for a prolonged period of time so as to release the active ingredient in a sustained fashion.

In contrast to other systems, the present invention provides an ophthalmically acceptable vehicle that not only has the benefit of administration in drop form, but also does not suffer from breakdown limitations due to administration at a viscosity suitable for drops. Through administration at a viscosity such that the suspension can be reliably administered in drop form, but which actually increases when the suspension is so administered, controlled release of the active ingredient is significantly enhanced.

A viscosity substantially over 30,000 cps is not useful for drop formulations; when the viscosity is substantially lower than about 1,000 cps, the ability to gel upon contact with tears can be impeded and ocular retention is reduced. The increased gelation upon contact with the tears occurs with a pH change when a suspension having a pH of from about 3 to about 7.4 and an osmolality of from about 10 to about 400 mOsm/kg, contacts tear fluid, which has a higher pH of about 7.2 to about 8.0. Without being bound by the theory, with the pH increase, the carboxylic acid (COOH) functional group disassociates into carboxylate anions (COO⁻). Through electrostatic interactions, these carboxylate ions repel each other, causing the polymer to expand. The presence of the trace second polymer in the system can provide additional electrostatic, hydrogen bonding, and possible salt-bridge interactions with the mucins of the ocular mucosa, in addition to providing the initial beneficial viscosity modifying properties to the base vehicle. These chemical interactions result in enhanced controlled release of medicament from the vehicle.

The relationship of cross-linking and particle size can be significant. Because the particles are present in a suspension, the degree of cross-linking is necessarily at a level that avoids substantial dissolution of the polymer. On the other hand, since rapid gelation is achieved at the time of the pH change, the degree of cross-linking is necessarily not so great that gelation is precluded. Moreover, if the polymer particle size is too large, induced swelling can tend to take up voids in the volume between large particles that are in contact with one another, rather than the swelling tending to cause gelation.

In a suspension, particle size can be relevant to comfort. However, it has been found that in the system of the present invention, the small particle size and light cross-linking act synergistically to yield the observed rapid gelation when the pH is raised. Surprisingly, the use of particles greater than about 25 μm eliminates the observed gelation when the pH of the vehicle is increased. Moreover, at about the 25 μm size, there is also reasonably good eye comfort.

In some embodiments, the particles are not only subject to the upper size limits described above, but also to a narrow particle size distribution. Use of a monodispersion of particles, which aids in good particle packing, yields a maximum increased viscosity upon contact of the suspension with the tears and increases eye residence time. At least about 80% in some embodiments, at least about 90% in other embodiments, and at least about 95% in still other embodiments, of the particles should be within a no more than about 10 μm band of major particle size distribution, and overall (i.e., considering particles both within and outside such band) there should be no more than about 20%, in some embodiments, and no more than about 10%, in other embodiments, and no more than about 5%, in still other embodiments, fines (i.e., particles of a size below 1 μm. In some embodiments, the average particle size is lowered from an upper limit of about 25 μm, such as about 15 μm, and to even smaller sizes such as 6 μm, such that the band of major particle size distribution is also narrowed, for example to 5 μm. In some embodiments, sizes for particles within the band of major particle distribution are less than about 30 μm, less than about 20 μm in other embodiments, and from about 1 μm to about 5 μm in still other embodiments.

The lightly cross-linked polymers of acrylic acid or related alpha, beta-unsaturated carboxylic acids used in ophthalmically acceptable vehicle are well known in the art. In one embodiment such polymers are prepared from at least about 90%, or about 95%, or about 99.9% by weight, based on the total weight of monomers present, of one or more carboxyl-containing monoethylenically unsaturated monomers. Acrylic acid is a common carboxyl-containing monoethylenically unsaturated monomer, but other unsaturated, polymerizable carboxyl-containing monomers, such as methacrylic acid, ethacrylic acid, beta-methylacrylic acid (crotonic acid), cis-alpha-methylcrotonic acid (angelic acid), trans-alpha-methylcrotonic acid (tiglic acid), alpha-butylcrotonic acid, alpha-phenylacrylic acid, alpha-benzylacrylic acid, alpha-cyclohexylacrylic acid, beta-phenylacrylic acid (cinnamic acid), coumaric acid (o-hydroxycinnamic acid), umbellic acid (p-hydroxycoumaric acid), and the like can be used in addition to or instead of acrylic acid.

Such polymers are cross-linked by using a small percentage, i.e., less than about 5%, such as from about 0.5% or from about 0.1% to about 1%, and in other embodiments from about 0.2% to about 1%, based on the total weight of monomers present, of a polyfunctional cross-linking agent. Included among such cross-linking agents are non-polyalkenyl polyether difunctional cross-linking monomers such as divinyl glycol; 2,3-dihydroxyhexa-1,5-diene; 2,5-dimethyl-1,5-hexadiene; divinylbenzene; N,N-diallylacrylamide; N,N-diallylmethacrylamide and the like. Also included are polyalkenyl polyether cross-linking agents containing two or more alkenyl ether groupings per molecule, preferably alkenyl ether groupings containing terminal H₂C═C< groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053. Diolefinic non-hydrophilic macromeric cross-linking agents having molecular weights of from about 400 to about 8,000, such as insoluble di- and polyacrylates and methacrylates of diols and polyols, diisocyanate-hydroxyalxyl acrylate or methacrylate reaction products, and reaction products of isocyanate terminated prepolymers derived from polyester diols, polyether diols or polysiloxane diols with hydroxyalkylmethacrylates, and the like, can also be used as the cross-linking agents; see, e.g., Mueller et al. U.S. Pat. Nos. 4,192,827 and 4,136,250.

The lightly cross-linked polymers can be made from a carboxyl-containing monomer or monomers as the sole monoethylenically unsaturated monomer present, together with a cross-linking agent or agents. They can also be polymers in which up to about 40%, and in some embodiments, from about 0% to about 20% by weight, of the carboxyl-containing monoethylenically unsaturated monomer or monomers has been replaced by one or more non-carboxyl-containing monoethylenically unsaturated monomers containing only physiologically and ophthalmologically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat. No. 4,548,990 for a more extensive listing of such additional monoethylenically unsaturated monomers. In some embodiments, polymers are lightly cross-linked acrylic acid polymers wherein the cross-linking monomer is 2,3-dihydroxyhexa-1,5-diene or 2,3-dimethylhexa-1,5-diene.

Exemplary commercially available lightly cross-linked carboxy-containing polymers useful in the invention include, for example, polycarbophil (available, for example, from BF Goodrich, Cleveland, Ohio), a polyacrylic acid cross-linked with divinyl glycol. Without being bound by theory, this polymer benefits from its mucoadhesive properties which aid in increasing the residence time of the active ingredient in the eye. Other mucoadhesive polymers can be used in conjunction with, or in lieu of the lightly cross-linked polymers disclosed herein, for example, Carbopols such as 934P, 940, 941,976, 971P, 974P, 980, 981 or hyaluronic acid. The latter has been demonstrated to be an effective mucoadhesive polymer in ocular formulations (Saettone et al. Int. J. Pharm. 51: 203-212, (1989)).

The lightly cross-linked carboxy-containing polymers can be prepared by suspension or emulsion polymerizing the monomers, using conventional free radical polymerization catalysts, to a dry particle size of not more than about 5.0 μm in equivalent spherical diameter; e.g., to provide dry polymer particles ranging in size from about 1 to about 3.0 μm, and in other embodiments from about 3 to about 10 μm, in equivalent spherical diameter. In general, such polymers will range in molecular weight estimated to be about 100,000 to about 4,000,000, and in some embodiments, about 2,000,000,000 to about 4,000,000,000.

Aqueous suspensions containing polymer particles prepared by suspension or emulsion polymerization whose average dry particle size is appreciably larger than about 10 □m in equivalent spherical diameter are less comfortable when administered to the eye than suspensions otherwise identical in composition containing polymer particles whose equivalent spherical diameters are, on the average, below about 10 μm. Moreover, above the average 5.0 μm size, the advantage of substantially increased viscosity after administration is not realized. It has also been discovered that lightly cross-linked polymers of acrylic acid or the like prepared to a dry particle size appreciably larger than about 5.0 μm in equivalent spherical diameter and then reduced in size, e.g., by mechanically milling or grinding, to a dry particle size of not more than about 5.0 μm in equivalent spherical diameter do not work as well as polymers made from aqueous suspensions in the ophthalmic vehicle of the invention.

While not being bound by any theory or mechanism advanced to explain the functioning of this invention, one possible explanation for the difference of such mechanically milled or ground polymer particles as the sole particulate polymer present is that grinding disrupts the spatial geometry or configuration of the larger than 5.0 μm lightly cross-linked polymer particles, perhaps by removing uncross-linked branches from polymer chains, by producing particles having sharp edges or protrusions, or by producing ordinarily too broad a range of particle sizes to afford satisfactory delivery system performance. A broad distribution of particle sizes impairs the viscosity-gelation relationship. In any event, such mechanically reduced particles are less easily hydratable in aqueous suspension than particles prepared to the appropriate size by suspension or emulsion polymerization, and also are less able to gel in the eye under the influence of tear fluid to a sufficient extent and are less comfortable once gelled than gels produced in the eye using the aqueous suspensions of this invention. However, up to about, 40% by weight, e.g., from about 0% to over 20% by weight, based on the total weight of lightly cross-linked particles present, of such milled or ground polymer particles can be admixed with solution or emulsion polymerized polymer particles having dry particle diameters of not more than about 50 μm when practicing this invention. Such mixtures also provide satisfactory viscosity levels in the ophthalmically acceptable vehicle and in the in situ gels formed in the eye coupled with ease and comfort of administration and satisfactory sustained release of the active ingredient to the eye, particularly when such milled or ground polymer particles, in dry form, average from about 0.01 to about 30 μm, and in other embodiments, from about 1 to about 5 μm, in equivalent spherical diameter.

In some embodiments, the particles have a narrow particle size distribution within a 10 μm band of major particle size distribution which contains at least 80%, in other embodiments at least 90%, and in still other embodiments at least 95% of the particles. Also, there is generally no more than about 20%, and in other embodiments no more than about 10%, and in still other embodiments no more than about 5% particles of a size below 1 μm. The presence of large amounts of such fines has been found to inhibit the desired gelation upon eye contact. Apart from that, the use of a monodispersion of particles gives maximum viscosity and an increased eye residence time of the active ingredient in the ophthalmically acceptable vehicle for a given particle size. Monodisperse particles having a particle size of about 30 μm and below are present in some embodiments. Good particle packing is aided by a narrow particle size distribution.

The aqueous suspensions can contain amounts of lightly cross-linked polymer particles ranging from about 0.1% to about 6.5% by weight, and in other embodiments from about 0.5% to about 4.5% by weight, based on the total weight of the aqueous suspension. They can be prepared using pure, sterile water, such as deionized or distilled, having no physiologically or ophthalmologically harmful constituents, and are adjusted to a pH of from about 3.0 to about 6.5, and in other embodiments from about 4.0 to about 6.0, using any physiologically and ophthalmologically acceptable pH adjusting acids, bases or buffers, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylaminomethane), or the like and salts and buffers such as citrate/dextrose, sodium bicarbonate, ammonium chloride and mixtures of the aforementioned acids and bases.

The second polymer can be any polymer that can enhance the viscosity and mucoadhesive properties of the vehicle where the combination is greater than each individual polymer alone and is also ophthalmically acceptable. Numerous examples of ophthalmically acceptable polymers are disclosed in Wagh et al. Asian J. Pharmaceutics (2008), which is incorporated by reference herein in its entirety. Exemplary second polymers include, without limitation, hydroxyproplymethyl cellulose (HPMC), hydroxypropyl cellulose (HPC), methyl cellulose (MC), hydroxyethyl cellulose (HEC), polyacrylic acid (PAA), polyvinyl alcohol, carbomers, sodium hyaluronate, chitosan, cyclodextrins, polygalacturonic acid, polyitaconic acid, xyloglucan, xanthan gum, gellan gum, polyorthoesters, celluloseacetophthalate, poloxamer 407, polyethyleneimine, and polyethylene oxide. In some embodiments, the second polymer can be a neutral polymer, a cationic polymer, or a second anionic polymer

In particular embodiments, the second polymer can be a cationic polymer. Cationic polymers include any ophthalmically acceptable polyamine polymer capable of modulating the rheological and/or mucoadhesive properties of the vehicle. Such polyamines include, for example, poly-L-lysine (PLL), chitosan, a naturally occurring polysaccharide containing D-glucosamine, polyethyleneimine (PEI), and polyquaternium compounds that include but not limited to Polyquarternium 1, Polyquaternium 7, and Polyquarternium 10, without being bound by theory, a cationic polymer can impact the vehicle characteristics in at least two different ways. Firstly, the cationic polymer can enhance electrostatic interactions between the carrier and the negatively charged mucins of the corneal epithelium. Such an interaction can confer beneficial mucoadhesive properties to the vehicle. Secondly, the viscosity of the aqueous suspension of the carboxy-containing polymer is increased by the addition of a cationic polymer, even prior to administration to the eye. Again, without being bound by theory, the cationic polyamine polymer can assist in particle aggregation through hydrogen bonding and/or by electrostatic interactions to effectively generate larger molecular weight constructs which increase the aqueous suspension's viscosity. In order to realize the benefits of the added cationic polymer, it should present in an amount that allows the particles of the carboxy-containing polymer to remain suspended, since these advantages are lost upon removal of the carboxy-containing particles from a suspended state. The increased viscosity of the dual cationic polymer/carboxy-containing polymer system can also help counter the effects of the clearance mechanisms in the eye.

In some embodiments, the cationic polymer is chitosan. Chitosan is obtained by deacetylation of chitin and possesses mucoadhesive properties due to electrostatic interaction between positively charged chitosan ammonium groups and negatively charged mucosal surfaces. Chitosan is a linear polysaccharide composed of randomly distributed β-(1-4)-linked D-glucosamine and N-acetyl-D-glucosamine. Chitosan is available with varying degrees of deacetylation (% DA) and is generally produced in a range from in a range from about 60 to about100% deacetylation. The amino group in chitosan has a pKa value of about 6.5, thus, chitosan is positively charged and soluble in acidic to neutral solution with a charge density dependent on pH and the % DA-value. Chitosan can enhance the transport of polar drugs across epithelial surfaces, and is considered biocompatible and biodegradable.

In some embodiments, chitosan used in the vehicle has a molecular weight in a range from in a range from about 50 kDa to about 100 kDa, including any weights in between, while in other embodiments, chitosan used in the vehicle has a molecular weight in a range from in a range from about 1,000 to about 3,000 kDa, and any weights in between. As shown in the Examples below, the range in a range from about 1,000 kDa and about 3,000 kDa appears to have a larger impact on viscosity of the vehicle, even at very small concentrations of the cationic polymer. In order to achieve comparable viscosities with chitosan alone, solutions of chitosan several orders of magnitude more concentrated have been used, for example, from in a range from about 2% to about 4%.

In the ophthalmically acceptable vehicle of the present invention, chitosan or other second polymer is present in an amount ranging from in a range from about 0.01% to about 0.5% when using a cationic polymer having a molecular weight ranging from about 50 kDa to about 100 kDa. The amount of cationic polymer or chitosan can be any amount in between, including about 0.01%, 0.025%, 0.05%. 0.075%, 0.10%, 0.15%, 0.20%, 0.25%, 0.30%, 0.35%, 0.40%, 0.45%, and 0.50% and any amount in between these values. When using higher molecular weight cationic polymers, such as in a range from about 1,000 to about 3,000 kDa, the amount of cationic polymer necessary to achieve favorable viscosities can be substantially reduced. For example, the amount of 1,000 kDa to about 3,000 kDa chitosan can be in a range in a range from about 0.01% and 0.5%, or any amount in between including, for example, 0.01%, 0.015%, 0.020%, 0.025%, 0.030%, 0.035%, 0.040%, 0.045%, 0.05%, 0.1%, 0.15%, 0.20%, 0.25%, 0.30%, 0.35%, 0.40%, 0.45%, and 0.50%.

When formulating the aqueous suspensions of this invention, their osmolality will be adjusted to from about 10 mOsm/kg to about 400 mOsm/kg, and in other embodiments, from about 100 to about 300 mOsm/kg, using appropriate amounts of physiologically and ophthalmologically acceptable salts. Sodium chloride can be used as an osmolality adjusting agent to adjust the osmolality of the aqueous suspension to approximate that of physiologic fluid. The amounts of sodium chloride ranging from about 0.01% to about 1% by weight, and in other embodiments from about 0.05% to about 0.45% by weight, based on the total weight of the aqueous suspension, will give osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like can also be used in addition to or instead of sodium chloride to achieve osmolalities within the above-stated ranges.

The amounts of lightly cross-linked carboxy-containing polymer particles, cationic polymer, the pH, and the osmolality chosen from within the above-stated ranges can be correlated with each other and with the degree of cross-linking to give aqueous suspensions having viscosities ranging from about 1,000 to about 30,000 cps, and in other embodiments from about 5,000 to about 20,000 cps, as measured at room temperature (about 25° C.) using a Brookfield Digital LVT Viscometer equipped with a number 25 spindle and a 13R small sample adapter at 12 rpm. The correlations of those parameters are also such that the suspensions will gel on contact with tear fluid to give gels having viscosities estimated to range from about 75,000 to about 500,000 cps, e.g., from about 200,000 to about 300,000 cps, measured as above, depending on pH as observed, for example, from pH-viscosity curves. This effect is noted by observing a more viscous drop on the eye as a set cast. The cast, after setting, can be easily removed. Alternatively, the viscosity can be from about 1000 to about 5000 cps as measured with a Brookfield cone and plate viscometer DV-II+ with the spindle no. CP-52 at 6 rpm.

In some embodiments, the viscosity is in a range from about 1,000 to about 30,000 cps, and in other embodiment from about 5,000 to about 20,000 cps. In yet other embodiments, the viscosity is in a range from about 10,000 to about 15,000 cps. The viscosity range can also be in a range from about 1,000 and 5,000 cps, including 1,000, 1,500, 2,000, 2,500, 3,000, 3,500, 4,000, 4500, and 5,000 cps and all values in between. The viscosity range can also be in a range from about 5,000 to about 10,000 cps, including 5,000, 5,500, 6,000, 6,500, 7,000, 7,500, 8,000, 8,500, 9,000, 9,500, and 10,000 cps and all values in between. The viscosity range can also be in a range from about 10,000 to about 15,000 cps, including 10,000, 10,500, 11,000, 11,500, 12,000, 12,500, 13,000, 13,500, 14,000, 14,500, and 15,000 cps and all values in between. The viscosity range can also be in a range from about 15,000 to about 20,000 cps, including 15,000, 15,500, 16,000, 16,500, 17,000, 17,500, 18,000, 18,500, 19,000, 19,500, and 20,000 cps and all values in between. The viscosity range can also be in a range from about 20,000 to about 30,000 cps, including 20,000, 21,000, 22,000, 23,000, 24,000, 25,000, 26,000, 27,000, 28,000, 29,000, and 30,000 cps and all values in between. In some embodiments, the ophthalmically acceptable vehicle can include a thickening agent or viscosfier that modulates the viscosity of the vehicle. These include, without limitation, polyethylene glycols, polyvinyl alcohol, polyacrylic acid, polyethylene oxide, and poloxamers.

In some embodiments, the present invention provides a composition that includes the ophthalmically acceptable vehicles described herein along with a medicament for treatment of a disease or disorder, wherein ocular delivery of the medicament is indicated for the treatment of said disease or disorder. Such compositions can also include two or more medicaments that can be used in a combination therapy as discussed further below. Ocular delivery can be indicated for diseases and disorder of the eye and surrounding tissues. One skilled in the art will also recognize the ability to deliver a drug systemically through an ocular route. Such systemic delivery can be useful to treat diseases or disorders beyond the eye itself and its surrounding tissues.

The viscous gels that result upon administration of the aqueous suspensions of this invention to the eye have residence times in the eye ranging from about 2 to about 12 hours, e.g., from about 3 to about 6 hours. The active ingredients contained in these ophthalmically acceptable vehicles are released from the gels at rates that depend on such factors as the active ingredient itself and its physical form, the extent of drug loading and the pH of the system, as well as on any drug delivery adjuvants, such as ion exchange resins compatible with the ocular surface, which can also be present. For fluorometholone, for example, release rates in the rabbit eye in excess of four hours, as measured by fluorometholone contained in the aqueous humor, have been observed.

The active ingredient-ophthalmically acceptable vehicle can be formulated in any of several ways. For example the active ingredient, lightly cross-linked polymer particles, and osmolality-adjusting agent can be pre-blended in dry form, added to all or part of the water, and stirred vigorously until apparent polymer dispersion is complete, as evidenced by the absence of visible polymer aggregates. Sufficient pH adjusting agent is then added incrementally to reach the desired pH, and more water to reach 100 percent formula weight can be added at this time, if necessary. Another convenient method involves adding the drug to about 95 percent of the final water volume and stirring for a sufficient time to saturate the solution. Solution saturation can be determined in any known manner, e.g., using a spectrophotometer. The lightly cross-linked polymer particles and the osmolality-adjusting agent are first blended in dry form and then added to the drug-saturated suspension and stirred until apparent polymer hydration is complete. Following the incremental addition of sufficient pH adjusting agent to reach the desired pH, the remainder of the water is added, with stirring, to bring the suspension to 100 percent formula weight.

These aqueous suspensions can be packaged in preservative-free, single-dose non-reclosable containers. This permits a single dose of the active ingredient to be delivered to the eye one drop at a time, with the container then being discarded after use. Such containers eliminate the potential for preservative-related irritation and sensitization of the corneal epithelium, as has been observed to occur particularly from ophthalmic medicaments containing mercurial preservatives. Multiple-dose containers can also be used, if desired, particularly since the relatively low viscosities of the aqueous suspensions of this invention permit constant, accurate dosages to be administered dropwise to the eye as many times each day as necessary.

In those vehicles where preservatives are to be included, suitable preservatives are chlorobutanol, Polyquat, benzalkonium chloride, cetyl bromide, benzethonium chloride, cetyl pyridinium chloride, benzyl bromide, EDTA, phenylmercury nitrate, phenylmercury acetate, thimerosal, merthiolate, acetate and phenylmercury borate, chlorhexidine, polymyxin B sulphate, methyl and propyl parabens, phenylethyl alcohol, quaternary ammonium chloride, sodium benzoate, sodium proprionate, sorbic acid, and sodium perborate. In particular embodiments, the preservative includes benzalkonium chloride.

In some embodiments, the preservative is present in a range from about 0.001 to about 0.02% by weight. The preservative can be present at about 0.001, 0.002, 0.003, 0.004, 0.005% and any amount in between these amounts. In particular, the present invention has the benefit of substantial reduction in the use of a bactericidal component. Thus, in some embodiments, the present invention provides an ophthalmically acceptable vehicle having less than about 0.01% of a preservative with bactericidal activity in one embodiment, and less than about0.01%, 0.009%, 0.008%, 0.007%, 0.006%, 0.005%, 0.004%, 0.003%, or 0.002%, in other embodiments.

In some embodiments, the ophthalmically acceptable vehicle includes a wetting agent. Such agents can be useful in distributing the active ingredient in an otherwise predominantly aqueous environment. Such wetting agents include, for example, Poloxamer 407, a triblock copolymer consisting of a central hydrophobic block of polypropylene glycol flanked by two hydrophilic blocks of polyethylene glycol. Other wetting agents that can be used include carboxymethylcellulose, hydroxypropyl methylcellulose, glycerin, mannitol, polyvinyl alcohol, Octoxynol 40 and hydroxyethylcellulose.

The composition containing a medicament and an ophthalmically acceptable vehicle can be individually packaged for a single dose administration; e.g., in a bottle, jar, ampoule, tube, syringe, envelope, container, unit dose container or vial. When the composition is individually packaged, in some embodiments, the composition does not include a preservative. Alternatively, the composition can be contained in a package that is capable of holding multiple units; e.g., in resealable glass or plastic eyedropper bottles.

In an embodiment, according to any of the above aspects of the invention, the invention relates to a composition or method for combination therapy of the eye of a mammal including: an ophthalmic composition having a therapeutically effective amount of bromfenac and dexamethasone, and a flowable mucoadhesive polymer such as DuraSite® or modified DuraSite as described above, wherein the composition has a viscosity formulated for administration to the eye of a mammal in drop form. In another embodiment, the invention relates to a composition or method for combination therapy of the eye of a mammal including: an ophthalmic composition having a therapeutically effective amount of bromfenac and dexamethasone, in an ophthalmic vehicle comprising a flowable mucoadhesive polymer such as DuraSite® or modified DuraSite® and one or more additional non-steroidal anti-inflammatory agent such as, for example, ketorolac. In another embodiment, the invention relates to a composition or method for combination therapy of the eye of a mammal including: an ophthalmic composition having a therapeutically effective amount of bromfenac and dexamethasone, a flowable mucoadhesive polymer such as DuraSite® or modified DuraSite® and one or more additional steroidal anti-inflammatory agents. In another embodiment, the invention relates to a composition or method for combination therapy of the eye of a mammal including: an ophthalmic composition having a therapeutically effective amount of bromfenac and dexamethasone, a flowable mucoadhesive polymer such as DuraSite® or modified DuraSite® and one or more antibacterial agent. In another embodiment, relating to any of the above aspects, the invention relates to a composition or method for combination therapy of the eye of a mammal including: an ophthalmic composition having a therapeutically effective amount of bromfenac and dexamethasone, a flowable mucoadhesive polymer such as DuraSite® or modified DuraSite® and an additional therapeutically active agent selected from the group consisting of antibacterial antibiotic agent, synthetic antibacterial agent, antifungal antibiotic agent, synthetic antifungal agent, antineoplastic agent, a second steroidal anti-inflammatory agent, a second non-steroidal anti-inflammatory agent, anti-allergic agent, glaucoma-treating agent, antiviral agent and anti-mycotic agent.

In some embodiments, according to any of the above aspects of the invention, the compositions of the invention can include, in addition to bromfenac and dexamethasone, one or more other active ingredients such as other NSAIDs. Suitable NSAIDs for combination therapy are, for example, aspirin, benoxaprofen, benzofenac, bucloxic acid, butibufen, carprofen, cicloprofen, cinmetacin, clidanac, clopirac, diclofenac, diflupredinate, etodolac, fenbufen, fenclofenac, fenclorac, fenoprofen, fentiazac, flunoxaprofen, furaprofen, flurbiprofen, furobufen, furofenac, ibuprofen, ibufenac, indomethacin, indoprofen, isoxepac, ketorolac, ketroprofen, lactorolac, lonazolac, metiazinic, miroprofen, nepafenac, naproxen, norketotifen, oxaprozin, oxepinac, phenacetin, pirprofen, pirazolac, protizinic acid, sulindac, suprofen, tiaprofenic acid, tolmetin, and zomepirac.

Unless the intended purpose of use is affected adversely, the ophthalmic formulation of the present invention can further comprise one or more additional therapeutically-active agents. Specific therapeutically-active agents include, but are not limited to: antibacterial antibiotics, synthetic antibacterials, antifungal antibiotics, synthetic antifungals, antineoplastic agents, further steroidal anti-inflammatory agents, further non-steroidal anti-inflammatory agents, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the invention and may be used in place of an agent itself.

Examples of the antibacterial antibiotics include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), .beta.-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefmetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Examples of the synthetic antibacterials include, but are not limited to: 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine-t, n.sup.2-formylsulfisomidine, n.sup.4-.beta.-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, n.sup.4-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

Examples of further steroidal anti-inflammatory agents include, but are not limited to: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Examples of the antifungal antibiotics include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrroInitrin, siccanin, tubercidin, viridin). Examples of the synthetic antifungals include, but are not limited to: allylamines (e.g., butenafine, naftifine, terbinafine), imidazoles (e.g., bifonazole, butoconazole, chlordantoin, chlormiidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

In general, ophthalmic formulations suitable for topical ophthalmic administration can be formulated and administered in accordance with techniques familiar to persons skilled in the art. The finished formulations are stored in opaque or brown containers to protect them from light exposure, and under an inert atmosphere. These compositions can be packaged in preservative-free, single-dose non-reclosable/reclosable containers or kits. This permits a single dose of the medicament to be delivered to the eye as a drop, with the container then being discarded after use. Such containers eliminate the potential for preservative-related irritation and sensitization of the corneal epithelium, as has been observed to occur particularly from ophthalmic medicaments containing mercurial preservatives. Multiple dose containers can also be used, if desired, particularly since the relatively low viscosities of the compositions of this invention permit constant, accurate dosages to be administered dropwise to the eye as many times each day as necessary. In those suspensions where preservatives are to be included, suitable preservatives are chlorobutanol, polyquat, benzalkonium chloride, cetyl bromide, sorbic acid and the like.

Another embodiment of the present invention includes the method of treating ocular inflammation and/or pain in a patient in need with one of the ophthalmic formulations described above, wherein said inflammation and/or pain is caused by surgical trauma, and wherein said treatment comprises treating the patient once or twice a day. An additional embodiment of the present invention includes the method of treating ocular pain and/or inflammation in a patient in need thereof, wherein said inflammation and accompanying pain is the result of cataract surgery or one of many refractive eye surgical techniques, and wherein said treatment comprises treating the patient once or twice daily with a formulation herein.

An additional embodiment of the present invention includes the method of treating ocular pain and/or inflammation in a patient in need thereof, wherein said inflammation and accompanying pain is the result of allergic, viral or bacterial conjunctivitis, and wherein said treatment comprises treating the patient with any of the disclosed formulations.

An additional embodiment of the present invention includes a method of treating ocular pain and/or inflammation associated with allergic, viral or bacterial conjunctivitis with one of the topical ophthalmic formulations of the invention. An additional embodiment may include one or more additional active ingredients as part of the formulation, such additional actives may include, but are not limited to, antihistamines and/or antibacterials and/or antimicrobial compounds, to further assist with the treatment of the conjunctivitis condition.

An additional embodiment of the present invention includes a method for treating an eye wherein its normal condition has been disrupted or changed comprising administering to said eye one to six times daily the formulation or composition of the invention. An additional embodiment of the present invention includes a method for treating postoperative inflammation and/or pain in patients who have undergone cataract extraction comprising the once, twice or up to six times daily administration of a selected formulation into the effected eye.

For example, in one embodiment, the methods of the invention encompass a process for therapeutic treatment of an inflammatory condition of the eye in a mammal including: (a) providing an ophthalmic composition comprising bromfenac and dexamethasone, each in an amount of about 0.005% to about 0.5% by weight of the composition and a flowable mucoadhesive polymer in an amount of about 0.5% to about 1.5% by weight of the composition; (b) administering said composition to the eye of a mammal in need thereof to treat inflammation or inflammatory conditions of the eye. In a related embodiment, the ophthalmic composition further includes a therapeutically active agent selected from the group consisting of antibacterial antibiotic agent, synthetic antibacterial agent, antifungal antibiotic, synthetic antifungal agent, antineoplastic agent, steroidal anti-inflammatory agent, non-steroidal anti-inflammatory agent, anti-allergic agent, glaucoma-treating agent, antiviral agent and anti-mycotic agent.

The inflammatory conditions for which the compositions and methods of the invention can be use are, but not limited to, surgical trauma; dry eye; allergic conjunctivitis; viral conjunctivitis; bacterial conjunctivitis; blepharitis; anterior uveitis; injury from a chemical; radiation or thermal burn; injury from penetration of a foreign body, pain in or around the eye, redness especially accompanied by pain in the eye; light sensitivity; seeing halos (colored circles or halos around lights); bulging (protrusion) of the eye; swelling of eye tissues; discharge, crusting or excessive tearing; eyelids stuck together, blood inside the front of the eye (on the colored part) or white of the eye; cataracts; pain and inflammation associated with wearing contact lenses; corneal-associated condition; conjunctival tumor excision; conjunctivitis known as Pink Eye; cornea edema after cataract surgery; corneal clouding; corneal transplantation; corneal ulcer; dry eye syndrome; dystrophies; condition associated with excimer laser phototherapeutic keratectomy; herpes simplex keratitis; keratoconus; pterygium; recurrent erosion syndrome; eye movement disorder; glaucoma; ocular oncology; oculoplastic condition resulted from cosmetic surgery, enucleation, eyelid and orbit injuries, ectropion, entropion, graves' disease, involuntary eyelid blinking; condition associated with refractive surgery; and retinal condition.

The retinal conditions for which the compositions and methods of the invention can be used are, but not limited to, macular degeneration, AIDS-related ocular disease, CMV retinitis, birdshot retinochoroidopathy (BR), choroidal melanoma, coats disease, cotton wool spots, diabetic retinopathy diabetic macular edema, cystoid macular edema, lattice degeneration, macular disease, macular degeneration, hereditary macular dystrophy, macular edema, macular hole, macular pucker, central serous chorioretinopathy, ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis of retina or uveitis.

In order that those skilled in the art can more fully appreciate aspects of this invention, the following Tables and examples are set forth. These examples are given solely for purposes of illustration and should not be considered as expressing limitations.

### EXAMPLE I

This Example shows the preparation of exemplary formulations, in accordance with some embodiments of the present invention.

TABLE 1

| Component | 1 | 3 | 3 | 4 | 5 | 6 | 7 | 8 |
|---|---|---|---|---|---|---|---|---|
| Polycarbophil | 0.9 | 0.9 | 0.9 | 0.9 | 0.9 | 0.9 | 0.9 | 0.9 |
| Bromfenac | 0.09 | 0.075 | 0.01 | 0.04 | 0.075 | 0.04 | 0.075 | 0.075 |
| Dexamethasone | 0.1 | 0.1 | 0.01 | 0.01 | 0.05 | 0.55 | 0.05 | 0.1 |
| Poloxamer 407 | 0.2 | 0.2 | 0.2 | 0.2 | 0.2 | 0.2 | 0.2 | 0.2 |
| Sodium Edetate | 0.1 | 0.1 | 0.1 | 0.1 | 0.1 | 0.1 | 0.1 | 0.1 |
| Sodium Citrate | 0.2 | 0.2 | 0.2 | 0.2 | 0.2 | 0.2 | 0.2 | 0.2 |
| Citric Acid | 0.14 | 0.14 | 0.14 | 0.14 | 0.14 | 0.14 | 0.14 | 0.14 |
| Boric acid | 0.49 | 0.49 | 0.49 | 0.49 | 0.49 | 0.49 | 0.49 | 0.49 |
| Sodium Borate | 0.51 | 0.51 | 0.51 | 0.51 | 0.51 | 0.51 | 0.51 | 0.51 |
| Sodium Chloride | 0.25 | 0.25 | 0.25 | 0.25 | 0.25 | 0.25 | 0.025 | 0.025 |
| Mannitol | — | — | — | — | — | — | 1.0 | 1.0 |
| Benzalkonium | 0.003 | 0.003 | 0.003 | 0.003 | 0.003 | 0.003 | — | — |
| chloride | | | | | | | | |
| Sodium | qs to 8.3 | qs to 8.3 | qs to 8.3 | qs to 8.3 | qs to 8.3 | qs to 8.3 | qs to 8.3 | qs to 8.3 |
| Hydroxide | | | | | | | | |
| Water, USP | qs to 100% | qs to 100% | qs to 100% | qs to 100% | qs to 100% | qs to 100% | qs to 100% | qs to 100% |

Formulations 1-8 in Table 1 are made by adding polycarbophil, sodium chloride and edetate to water by stirring for 0.5 hours. The solution is then sterilized at 121° C. for 45 minutes and cooled to room temperature. The citrate buffer is dissolved in water and added by sterile addition through a 0.2 um filter while mixing. The mannitol, poloxamer, and bromfenac are dissolved in water and added to the batch by sterile addition. The dexamethasone which has been sterilized by Co-60 radiation is added to the batch by sterile dry particle addition and mixed into the batch. The borate buffer and benzalkonium chloride are dissolved and added by sterile filtration while mixing. Sodium hydroxide is added by stile addition to adjust the pH to 8.3.

The embodiments within the specification provide an illustration of embodiments of the invention and should not be construed to limit the scope of the invention. The skilled artisan readily recognizes that many other embodiments are encompassed by the invention.

We claim:

1. An ophthalmic composition comprising a therapeutically effective amount of bromfenac, a therapeutically effective amount of a steroidal anti-inflammatory, and an opthalmically acceptable vehicle comprising a flowable mucoadhesive polymer, wherein the composition has a viscosity formulated for administration to the eye of a mammal in drop form.

2. The ophthalmic composition according to claim 1, wherein bromfenac is present in a range from about 0.01% to about 0.25% by weight of the composition.

3. The ophthalmic composition according to claim 1, wherein the steroidal anti-inflammatory is a glucocorticoid.

4. The ophthalmic composition according to claim 3, wherein the glucocorticoid comprises one selected from the group consisting of hydrocortisone, cortisone acetate, prednisone, prednisolone, methylprednisolone, dexamethasone, betamethasone, triamcinolone, beclomethasone, and fluorometholone.

5. The ophthalmic composition according to claim 4, wherein the glucocorticoid is dexamethasone in a form comprising at least one selected from the group consisting of dexamethasone sodium phosphate, dexamethasone (alcohol), dexamethasone acetate, dexamethasone dimethylbutyrate, dexamethasone trimethylacetate, dexamethasone dipropionate, dexamethasone acefurate, and mixtures thereof.

6. The ophthalmic composition according to claim 1, wherein the steroidal anti-inflammtory is present in a range from about 0.025% to about 0.25% by weight of the compostion.

7. The ophthalmic composition according to claim 1, wherein the flowable mucoadhesive polymer is a lightly crosslinked carboxy-containing polymer.

8. The ophthalmic composition according to claim 7, wherein the carboxy-containing polymer is polycarbophil.

9. The ophthalmic composition according to claim 1, wherein the flowable mucoadhesive polymer is in an amount of about 0.5% to about 1.5% by weight of the composition.

10. The ophthalmic composition according to claim 1, wherein the composition has a pH of about 7.4 to about 8.5.

11. The ophthalmic composition according to claim 1, wherein the viscosity of the composition is in the range of about 1,000 to about 2,000 cps.

12. The ophthalmic composition according to claim 1, wherein the ophthalmically acceptable vehicle further comprises a second polymer comprising a cationic polymer.

13. The ophthalmic composition according to claim 12, wherein the viscosity of the composition is in the range of about 1,000 to about 30,000 cps.

14. The ophthalmic composition according to claim 1, wherein the composition further comprises an additional therapeutically active agent selected from the group consisting of antibacterial antibiotic agent, synthetic antibacterial agent, antifungal antibiotic agent, synthetic antifungal agent, antineoplastic agent, anti-allergic agent, glaucoma-treating agent, antiviral agent and anti-mycotic agent.

15. A method for therapeutic treatment of an inflammatory condition of the eye in a mammal comprising: (a) providing an ophthalmic composition comprising a therapeutically effective amount of bromfenac, a therapeutically effective amount of a steroidal anti-inflammatory, and an opthalmically acceptable vehicle comprising a flowable mucoadhesive polymer, wherein the composition has a viscosity formulated for administration to the eye of a mammal in drop form; and (b) administering said composition to the eye of a mammal to treat inflammation or inflammatory conditions of the eye.

16. The method according to claim 15, wherein the ophthalmic composition further comprises a therapeutically active agent selected from the group consisting of an antibacterial antibiotic agent, a synthetic antibacterial agent, an antifungal antibiotic, a synthetic antifungal agent, an antineoplastic agent, an anti-allergic agent, a glaucoma-treating agent, an antiviral agent and an anti-mycotic agent.

17. The method to claim 15, wherein the inflammatory condition is selected from the group consisting of surgical trauma; dry eye; allergic conjunctivitis; viral conjunctivitis; bacterial conjunctivitis; blepharitis; anterior uveitis; injury from a chemical; radiation or thermal burn; injury from penetration of a foreign body, pain in or around the eye, redness especially accompanied by pain in the eye; light sensitivity; seeing halos (colored circles or halos around lights); bulging (protrusion) of the eye; swelling of eye tissues; discharge, crusting or excessive tearing; eyelids stuck together, blood inside the front of the eye (on the colored part) or white of the eye; cataracts; pain and inflammation associated with wearing contact lenses; corneal-associated condition; conjunctival tumor excision; conjunctivitis known as Pink Eye; cornea edema after cataract surgery; corneal clouding; corneal transplantation; corneal ulcer; dry eye syndrome; dystrophies; condition associated with excimer laser phototherapeutic keratectomy; herpes simplex keratitis; keratoconus; pterygium; recurrent erosion syndrome; eye movement disorder; glaucoma; ocular oncology; oculoplastic condition resulted from cosmetic surgery, enucleation, eyelid and orbit injuries, ectropion, entropion, graves' disease, involuntary eyelid blinking; condition associated with refractive surgery; and retinal condition.

18. The method according to claim 15, wherein the inflammatory condition is a retinal condition.

19. The method according to claim 18, wherein the retinal condition is age related macular degeneration, AIDS-related ocular disease, CMV retinitis, birdshot retinochoroidopathy (BR), choroidal melanoma, coats disease, cotton wool spots, diabetic retinopathy diabetic macular edema, cystoid macular edema, lattice degeneration, macular disease, macular degeneration, hereditary macular dystrophy, macular edema, macular hole, macular pucker, central serous chorioretinopathy, ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis of retina or uveitis.
